(19) United States
(12) Patent Application Publication
(10) Pub. No.: US 2022/0133386 A1
(43) Pub. Date: May 5, 2022

(54) ELECTROSURGICAL SYSTEM WITH EVACUATION DEVICE

(71) Applicant: OLYMPUS WINTER & IBE GMBH, Hamburg (DE)

(72) Inventors: Fabian JANICH, Potsdam (DE); Frank BREITSPRECHER, Berlin (DE); Jens KRÜGER, Eichwalde (DE)

(73) Assignee: OLYMPUS WINTER & IBE GMBH, Hamburg (DE)

(21) Appl. No.: 17/487,185

(22) Filed: Sep. 28, 2021

(30) Foreign Application Priority Data
Oct. 30, 2020 (DE) 102020128693.7

(51) Int. Cl. A61B 18/12; A61B 18/08

(52) U.S. Cl. CPC A61B 18/1206; A61B 2018/00779; A61B 18/082

(57) ABSTRACT

An electrosurgical system with an electrosurgical instrument, an evacuation device and an electrosurgical generator, to which the electrosurgical instrument and the evacuation device are connected, wherein the electrosurgical generator has a processor and at least one memory and is configured to convert entries in the memory into control instructions for operating the evacuation device and, during operation, to carry out control interventions corresponding to the control instructions for controlling the evacuation device, wherein the entries in the memory defining the control instructions for operating the evacuation device are specific to a respective electrosurgical instrument and/or a respective operating mode.

The invention relates to an electrosurgical system with an electrosurgical generator that is configured to supply high-frequency alternating current to an electrosurgical instrument and to control an evacuation device. The invention also relates to an electrosurgical instrument and an electrosurgical generator.

An electrosurgical system generally comprises an electrosurgical generator for generating the high-frequency alternating current. As a general rule, the electrosurgical generator has two or more outputs where an electrosurgical instrument can be connected, and a high-frequency AC voltage is provided between these outputs during operation.

Typically, different electrosurgical instruments for different tasks, and an evacuation device, in particular a smoke evacuation device, can be connected to the electrosurgical generator.

Electrosurgery can be used for cutting, coagulating (obliterating) and/or vaporizing biological tissue, i. e. body tissue. High-frequency alternating currents with a frequency between 0.2 MHz and 3 MHz are typically used in electrosurgery. Electrosurgical instruments are typically handheld instruments that a surgeon can use to coagulate, ablate and/or cut body tissue.

To this end, the electrosurgical instruments are supplied with high-frequency electrical energy, by means of which tissue can be coagulated or cut in a targeted manner. The high-frequency electrical energy is supplied by the electrosurgical generator and applied to the body tissue by means of the electrosurgical instrument that is appropriate in the respective case. Depending on what the electrosurgical instrument is used for, specific current and voltage curves are required; these current and voltage curves are made available to the physician for selection at the electrosurgical generator in the form of operating modes (also referred to as modes). These operating modes can be permanently stored in the electrosurgical generator.

During the use of an electrosurgical instrument, smoke may be generated, for example, depending on the instrument and operating mode. Since these can be disturbing or even harmful under certain circumstances, known electrosurgical generators can be used in combination with an evacuation device, in particular a smoke evacuation device. The smoke evacuation device can be switched on and off, which is controlled by the electrosurgical generator.

It is the object of the invention to create an electrosurgical system that offers improved control of an evacuation device.

To this end, the invention proposes an electrosurgical system, an electrosurgical generator and an electrosurgical instrument which can each individually and in combination with each other allow for different operating modes that are tailored to a particular application and/or to a particular electrosurgical instrument, and allow for appropriate control of an evacuation device.

An electrosurgical system according to the invention comprises an electrosurgical instrument and an electrosurgical generator to which the electrosurgical instrument and an evacuation device are connected during operation.

The electrosurgical generator has a processor and at least one generator data memory in which an operating program for controlling the operation of the electrosurgical generator in combination with the electrosurgical instrument and the evacuation device is stored.

By means of the operating program, the processor is configured to read specific control instructions for different electrosurgical instruments and/or different operating modes and to cause their implementation into control interventions on the electrosurgical generator, the electrosurgical instrument and/or the evacuation device. In this context, control interventions comprise, for example, controlling the delivery of energy to an electrosurgical instrument that is, during operation, connected to the electrosurgical generator. However, control interventions can also be the switching on or off of the evacuation device, so that, for example, power control in the sense of pulse width modulation is possible by switching the evacuation device on and off in rapid succession. During operation, the electrosurgical generator thus converts control instructions into control interventions. For this purpose, the electrosurgical generator is controlled by the processor and the operating program stored in the generator data memory.

The invention thus proposes an electrosurgical system with an electrosurgical instrument, an evacuation device and an electrosurgical generator, to which the electrosurgical instrument and the evacuation device are connected, wherein the electrosurgical generator has a processor and at least one data memory and is configured to convert entries in the data memory into control instructions for operating the evacuation device and, during operation, to carry out control interventions corresponding to the control instructions for controlling the evacuation device, and wherein the entries defining the control instructions for operating the evacuation device are specific to a respective electrosurgical instrument and/or a respective operating mode.

The control instructions to be read by the processor during operation under the control of the operating program, which are specific to a respective electrosurgical instrument and/or a respective operating mode, can be defined directly or indirectly by entries stored in a generator data memory of the electrosurgical generator and/or an instrument data memory of the electrosurgical instrument. According to the invention, entries defining control instructions for control interventions on the evacuation device are in each case stored as assigned to an operating mode and/or an electrosurgical instrument. The control instructions defined by stored entries for control interventions on the evacuation device can be conditional or define control interventions dynamically dependent on respective current operating states of the electrosurgical generator or on currently occurring operating parameter values (such as the power effectively output by the electrosurgical instrument).

The generator data memory of the electrosurgical generator preferably contains a theoretical arbitrary number of operating specifications that can be called up by the operating program and influence the operation of the electrosurgical generator, but which do not define any fixed operating sequences. The operating specifications can either be control commands or parametric data, such as values for the output voltage, output current, suction output of the evacuation device, time settings, threshold values, error conditions, etc.

The operating specifications in the generator data memory can be understood as a library of any plurality of possible control instructions or parameter values that can be accessed by the operating program. However, according to the preferred embodiment, these operating specifications are not accessed directly, so that the operating specifications and the operating program as such do not determine the mode of operation of the electrosurgical generator.

Rather, a data structure, in which arbitrary data sets with structured data are stored, is preferably also provided. The structured data contains references to concrete operating specifications in the generator data memory, i.e. to concrete control commands and/or parameter values. The structured data contains the aforementioned entries that define the control instructions the processor converts, based on the operating program, into control interventions on the electrosurgical generator or evacuation device. These entries defining the control instructions are thus stored in the data structure.

For example, the control instructions defined by entries in the structured data that the electrosurgical generator converts into control interventions under the control of the processor in conjunction with the operating program, are defined by control commands and, if applicable, assigned parameter values. The references to operating specifications contained in the structured data are called up during operation of the electrosurgical generator by the then running operating program and, as a result, those operating specifications to which the references refer are applied. The processor controlled by the operating program applies the operating specifications and initiates appropriate control interventions by the electrosurgical generator and/or the evacuation device. The control interventions can thus involve both the respective electrosurgical instrument and the evacuation device.

The specific mode of operation of the electrosurgical generator in the preferred embodiment is thus dependent on three different types of stored data, namely
on the data in the generator data memory which defines the operating program;
on the potentially applicable operating specifications in the generator data memory, which define e.g. control commands or parameter values, and some of which are specifically applied because of the references contained in the data structure;
on the data sets with structured data stored in the data structure, which contain the references to specifically applicable operating specifications and which are called up by the processor during the course of the operating program and translated into specifically applicable operating specifications.

The structured data stored in the data structure preferably contains entries that define, either directly or by way of the references and in the data structure and the operating specifications in the generator data memory, control instructions that are converted into control interventions on the evacuation device during operation of the electrosurgical generator. The control instructions concerning the evacuation device can, for example, define an on or off switching of the evacuation device or, for the switched-on state of the evacuation device, the respective power to be delivered by the evacuation device.

The electrosurgical generator can be designed to convert such control instructions, which define the power to be delivered by the evacuation device, into control interventions in the form of pulse sequences which, in the sense of pulse width modulation, cause the evacuation device to be switched on and off in rapid succession so that it is operated with a higher or lower capacity as a result. A sequence of switch-on pulses emitted in rapid succession in the context of pulse width modulation is defined by the length of the switch-on pulses and the length of the pauses (switch-off times) between the individual switch-on pulses. The shorter the pauses (switch-off times) and the longer the switch-on pulses, the higher the power output of the evacuation unit is as a result.

In a preferred embodiment, the electrosurgical generator can be designed to control the power supplied by the evacuation device during operation depending on a calculated parameter value or a parameter value detected by sensors. Furthermore, the electrosurgical generator can have power measuring means for measuring a power output via an electrosurgical instrument so as to control the evacuation device according to the power output by the electrosurgical instrument.

Preferably, the control interventions during operation of the electrosurgical generator result from the operating program in combination with the operating specifications in the generator data memory and the references or control instructions in the data structure stored in either the instrument data memory or the generator data memory. The operating program, the operating specifications and the data structure with the references and control instructions can be changed and specified independently of each other, with the limitation that the data formats are compatible. Thus, the mode of operation of the electrosurgical generator in a respective operating mode can be changed by modifying the operating program stored in the generator data memory, or by modifying the operating specifications, or by modifying the structured data in the data structure, or also through a combination of these changes.

The electrosurgical instrument preferably contains a non-volatile instrument data memory (e.g. an EEPROM or similar), but no processor. The instrument data memory of the surgical instrument contains a data set with structured data. The structured data of this data set is compatible with the data structure in the generator data memory of the electrosurgical generator and may form the data structure of the electrosurgical generator or a part of the data structure of the electrosurgical generator. Preferably, the data structure stored in the non-volatile instrument data memory of the electrosurgical instrument includes entries that define the control instructions for control interventions on the evacuation device. This means that it is possible to define a control of the evacuation device during operation that is suitable for a particular electrosurgical instrument and, if applicable, for the respective operating mode.

The data structure containing entries for control instructions defining control interventions on the evacuation device containing can thus be part of a generator data memory, which is a physical component of the electrosurgical generator, or it is formed by the content of the instrument data memory of the electrosurgical instrument, or by a combination of contents in the generator data memory of the electrosurgical generator and the instrument data memory of the electrosurgical instrument.

Controlled by the operating program, the processor of the electrosurgical generator is configured to read the instrument data memory of the electrosurgical instrument in order to ensure, for example, that the evacuation device is controlled appropriately for the respective electrosurgical instrument during operation.

The processor of the electrosurgical generator can be configured, in combination with the operating program, to read the instrument data memory of the electrosurgical instrument after the electrosurgical instrument has been connected to the electrosurgical generator and before the electrosurgical generator is operated in an operating mode. The processor of the electrosurgical generator can be configured, in combination with the operating program, to transfer the data set or the data sets or the structured data contained in the data set or the data sets into a data structure that is part of the electrosurgical generator.

The processor of the electrosurgical generator can alternatively be configured, in combination with the operating program, to read the instrument data memory of the electrosurgical instrument while the operating program is running—i.e. while the electrosurgical generator is being operated in an operating mode. Thus, no computer program or algorithm is stored in the instrument data memory of the surgical instrument, and the structured data is neither readable as a computer program nor as an algorithm associated with a program.

The structure of the structured data makes it possible to assign line numbers to the references. With the help of the line numbers, the references associated with them can be called up in a targeted manner by means of the operating program. The references, in turn, unambiguously refer to specific operating specifications in the generator data memory or, alternatively, also to other references or control commands in the structured data.

Instead of providing line numbers that are explicitly stored in the data set, it may be provided that the line numbers are only generated when a data set is read, namely based on the structure of the structured data in a data set that represents the references. This is possible if the references are, for example, stored in a designated order in a data set.

The structured data is preferably available in a memory-efficient binary format.

The electrosurgical system preferably has a programming interface, or several programming interfaces, by means of which the contents of the data structure or generator data memory can be programmed. Accordingly, it may also be provided that the operating program in the generator data memory can be changed via a programming interface, for example by way of a software update of the electrosurgical generator via USB.

The invention also proposes a method for operating an electrosurgical generator, according to which operating specifications and a data structure are provided independently of one another, wherein the data structure contains references to the operating specifications, and a processor controlled by an operating program indirectly accesses individual operating specifications, while the operating program is running, by first accessing references in the data structure and subsequently retrieving that operating specification or those operating specifications to which a respective reference refers.

The data structure is preferably read from an instrument data memory of an electrosurgical instrument, and preferably after an electrosurgical instrument has been connected and before it is used. The data sets with structured data contained in the data structure on the electrosurgical instrument can be transferred into a data structure that is stored on an electrosurgical generator.

Another aspect of the invention is an electrosurgical generator for an electrosurgical system of the aforementioned type. The electrosurgical generator has connections for connecting an electrosurgical instrument as well as a processor and at least one generator data memory, in which
first data is stored that defines an operating program for controlling the operation of the electrosurgical generator in conjunction with the electrosurgical instrument;
second data is stored that defines the operating specifications that can be called up by the operating program and influence the operation of the electrosurgical generator, but which do not define any fixed operating sequences; and
third data, which defines a data structure containing data sets with structured data, contains references to operating specifications stored in the generator data memory.

Such an electrosurgical generator can easily be used with a variety of different electrosurgical instruments, wherein the adaptation to a respective electrosurgical instrument can be made solely by means of corresponding entries in the data structure which represent references or control instructions, without the operating specifications in the generator data memory of the electrosurgical generator having to be changed.

Preferably, in addition to the references stored in it, the data structure also contains line numbers assigned to these references, which allow for individual references to be specifically called up by the operating program during operation.

A further aspect of the invention is an electrosurgical instrument that has an instrument data memory containing data sets with structured data which contains references to operating specifications stored in the generator data memory. Such an electrosurgical instrument can therefore contain the necessary information that allows for an operating mode that is adapted to the respective electrosurgical instrument, without said operating mode having to be completely defined by the electrosurgical instrument.

The invention will now be explained in more detail based on exemplary embodiments referencing the figures. The figures show the following:

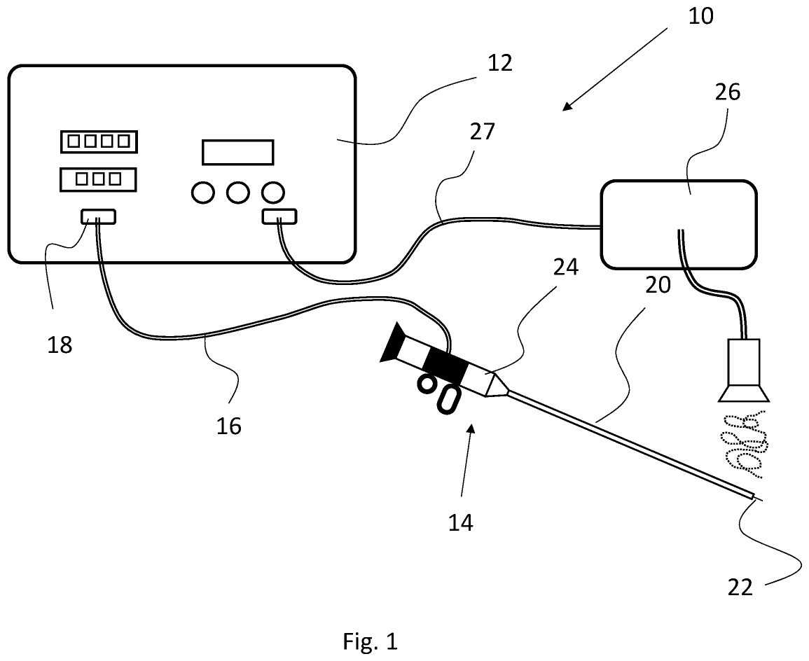

FIG. 1: an electrosurgical system with an electrosurgical generator and an electrosurgical instrument connected thereto;

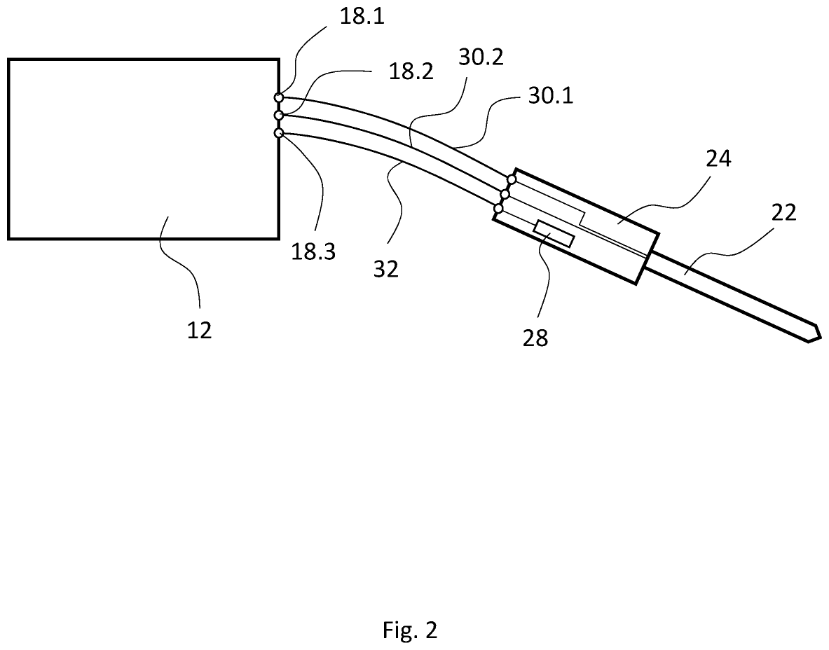

FIG. 2: an electrosurgical instrument;

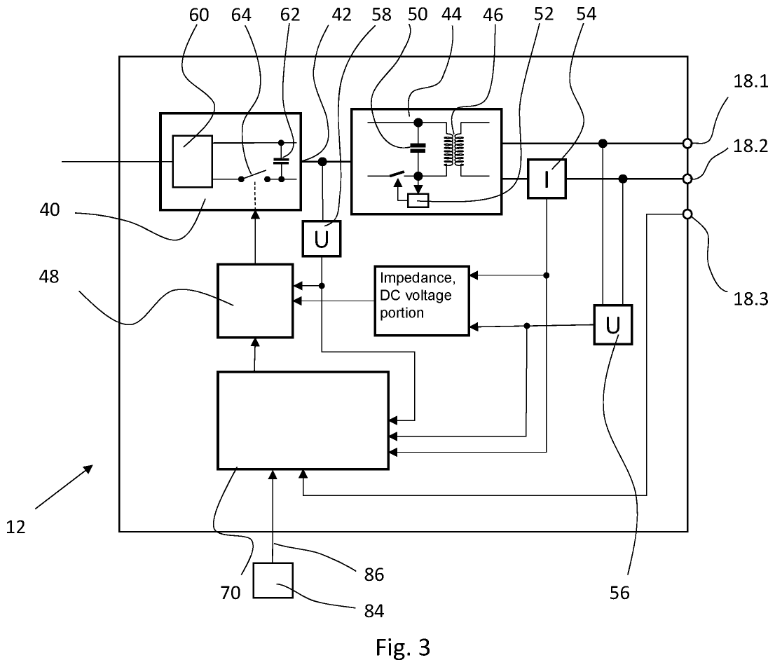

FIG. 3: a schematic diagram of an electrosurgical generator;

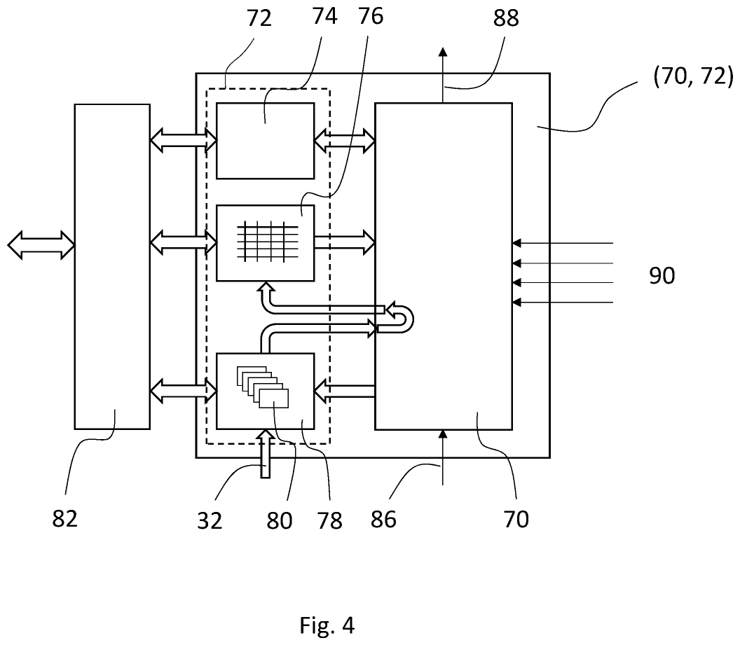

FIG. 4: a schematic illustration of a processor in combination with a generator data memory of the electrosurgical generator of FIG. 3; and

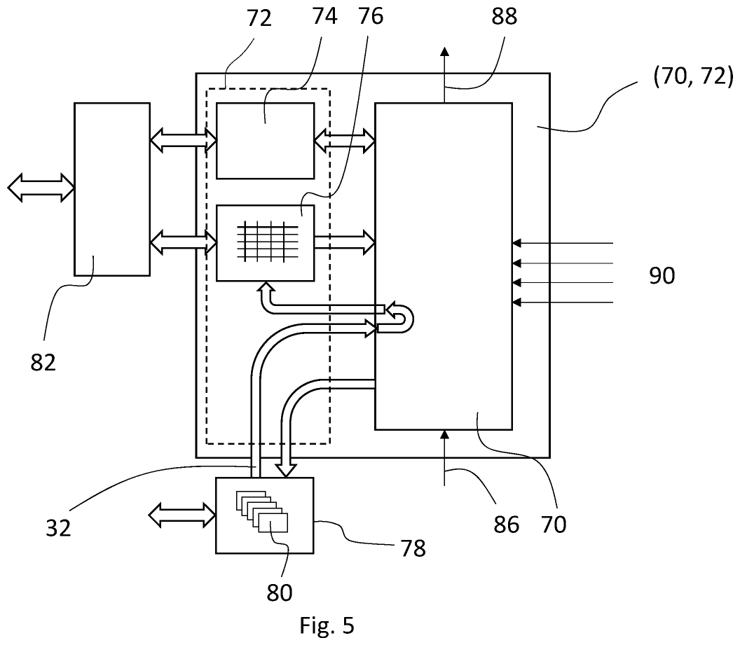

FIG. 5: a schematic illustration of an alternative configuration of the processor in combination with a generator data memory of the electrosurgical generator of FIG. 3.

FIG. 1 shows an electrosurgical system 10. The electrosurgical system 10 comprises an electrosurgical generator 12 and an electrosurgical instrument 14. Via a connection cable 16, the electrosurgical instrument 14 is connected to electrical outputs and inputs 18 of the electrosurgical generator 12.

The electrosurgical instrument 14 has a shaft 20, at the end of which is an active electrode 22. The shaft 20 is attached to a handle 24 of the electrosurgical instrument 14.

In addition, an evacuation device 26 for smoke evacuation is connected to the electrosurgical generator 12. Via a control line 27, the evacuation device 26 can be switched on and off under the control of the electrosurgical generator 12.

The electrosurgical instrument 14 features an instrument data memory 28 as a non-volatile data memory for data. The instrument data memory 28 is non-volatile and can be a ROM (Read Only Memory), for example, such as an EPROM (electrically programmable read-only memory), in particular an EEPROM (electrically erasable programmable read-only memory). An EEPROM is a non-volatile data memory that can be read, written, and write-protected. For example, the instrument data memory 28 is located in the handle 24 of the electrosurgical instrument 14.

The connection cable 16 contains both the supply lines 30.1 and 30.2 and at least one data line 32. The supply lines 30.1 and 30.2 connect the active electrode 22 and another neutral electrode, that is not described in more detail, to the electrical outputs 18.1 and 18.2 of the electrosurgical generator 12. Via the data line 32, the data memory 28 is connected to a corresponding connection 18.3 of the electrosurgical generator 12. This is shown schematically in FIG. 2.

The data line 32 in the connection cable 16 as well as the connection 18.3 can be a multicore and/or multi-pole line/connection. In addition to or instead of the data line 32, a wireless interface may be provided for the data transfer from the electrosurgical instrument 14 to the electrosurgical generator 12. Such a wireless interface may, for example, be a Bluetooth interface, an NFC interface or an RFID interface.

During operation, the AC output voltage that is to be supplied to the active electrode 22 and a return electrode of the electrosurgical instrument 14 for the operation of the electrosurgical instrument 14 is provided by the electrosurgical generator 12. As shown in FIG. 3, the electrosurgical generator 12 has a high-voltage power supply 40 for this purpose, which can be connected to the usual public power grid and provides a high-frequency direct current with DC output voltage at its output 42. This direct output current is supplied to a high-frequency part 44 of the electrosurgical generator 12. The high-frequency part 44 of the electrosurgical generator 12 serves as an inverter and produces a high-frequency AC output voltage that is supplied to the outputs 18.1 and 18.2 of the electrosurgical generator 12 via an output transformer 46 of the high-frequency part 44. The electrosurgical instrument 14 can be connected to the outputs 18.1 and 18.2 of the electrosurgical generator 12, as shown in FIGS. 1 and 2.

To control the AC output voltage of the electrosurgical generator 12, a generator control unit 48 is provided that controls the AC output voltage at the outputs 18.1 and 18.2 of the electrosurgical generator 12 based on a maximum AC output voltage value such that, for example, a preset maximum output voltage value is not exceeded during operation.

The AC output voltage of the electrosurgical generator 12—and therefore also the alternating output current and the output power—can be controlled through the DC output voltage generated by the high-voltage power supply.

This is the purpose of the generator control unit 48 that controls the DC output voltage generated by the high-voltage power supply 40 in such a way that a resulting AC output voltage or an alternating output current are the voltage and/or current required by the respective operating mode of the electrosurgical generator at a respective point in time.

Each operating mode defines maximum values for the RMS of the AC output voltage through the outputs 18.1 and 18.2, the peak output voltage through the outputs 18.1 and 18.2, the RMS of the alternating output current at the outputs 18.1 or 18.2, the DC voltage portion of the AC output voltage through the outputs 18.1 and 18.2 as well as the DC output voltage of the high-voltage power supply 40. The maximum values defined by a respective operating mode may be situation-dependent and change during use.

The generator control unit 48 controls the high-voltage power supply 40 in dependence on maximum values defined by a respective operating mode and on values of the AC output voltage, the peak output voltage, the alternating output current, the DC voltage portion of the AC output voltage or the DC output voltage detected during operation by detection units 54, 56 and 58, or on a combination of values of these parameters.

The specific maximum values and the time sequence for the generation of the DC output voltage of the electrosurgical generator 12 and their dependence on detected momentary values depend on the respective operating mode in which the electrosurgical generator 12 is currently being operated.

An operating mode is, for example, called up through the actuation of a corresponding switch by a user, for example a foot-operated switch 84.

In a respective operating mode, the operation of the electrosurgical generator 12 is controlled by a processor 70 in combination with an operating program 74 stored in a generator data memory 72, to which the processor is connected. The processor 70 generates, in combination with the operating program 74, the maximum values for the different operating parameters, for example—such as the AC output voltage, the alternating output current, the output power, but also the DC voltage portion of the AC output voltage, wherein the respective current value of these parameters is detected during operation of the electrosurgical generator 12.

During execution of the operating program 74 stored in the generator data memory 72, the processor 70 accesses, at locations stored in the operating program, operating specifications 76, such as data representing values for operating parameters and/or control commands, which are also stored in the generator data memory 72 for a respective operating mode. The operating specifications 76 stored in the generator data memory 72 specify, for example, specific values for the DC output voltage of the high-voltage power supply or the AC output voltage, the alternating output current of the high-frequency part or similar data. However, operating specifications 76 stored in the generator data memory 72 also include specific control commands, such as “if” or “while”, or “true” or “false”. That way the data and control commands stored as operating specifications in the generator data memory 72 can, for example, be used to define control instructions such as “compare the current value of the AC output voltage to the amount 200 and return “true” if the current value of the voltage is smaller than or equal to 200 and “false” if the current value is greater than 200″. Other data and control commands can, for example, be combined with the control instruction stating that the maximum DC output voltage of the high-voltage power supply shall be 100 Volt.

However, in order to generate and execute such control instructions, the processor 70 does not access the operating specifications 76 in the generator data memory 72 directly, but calls up a data structure 78 at the respective points of the operating program 74; references referring to corresponding operating specifications 76 in the generator data memory 76 are stored for the respective operating mode in said data structure 78; cf. e.g. FIG. 4. The references have a size of 1-byte so that they require little memory space. In this case, the operating specifications 76 can be structured in the form of a table, in which each reference (i.e. for example each hexadecimal number) is assigned the corresponding control commands or data.

A plurality of data sets 80 that each contain one reference or several references which, due to the structure of the respective data set—in particular the order in which the references are stored—can be assigned line numbers, are stored in the data structure 78, so that different operating modes can be implemented. The references assigned to a line number refer to the corresponding operating specifications 76 in the generator data memory 72 and cause the processor 70 to read the corresponding operating specifications 76 from the generator data memory 72, after the processor 70 has first accessed the vector address stored in the data structure 78 and designated by the associated line number. The operating specifications stored in the generator data memory 72 may, for example, be control instructions, control commands or parameter values, which the operating program is to apply at the respective point of the operating program where the operating program 74 contains a reference to a line number in the data structure 78.

References to the operating specifications 76 stored in the generator data memory 72 are thus stored in the data structure 78 in an ordered sequence. The structure of a respective data set in the data structure 78 makes it possible to assign line numbers, like addresses, within the data structure to the references so that, for example, jumps or returns to references in the data structure 78 are possible and not only a strictly sequential processing of the references by the operating program. Line numbers can serve as vector addresses within the structured data in the data structure 78, which the processor 70 accesses under the control of the operating program 74. The references assigned to the line numbers refer to corresponding operating specifications 76 in the generator data memory 72. These operating specifications 76 are, for example, individual control instructions, composite control instructions, or parameter values for operating parameters. The operating specifications 76 called up by the parameterized references in the data structure 78 control the operation of the electrosurgical generator 12 in the respective operating mode in combination with the operating program 74. The operating specifications may also be control commands that cause other references in the data structure 78 to be called up. However, this is only possible within the data—i.e. within the references that belong to a respective operating mode.

The references are preferably represented by hexadecimal numbers that together form structured data of a data set 80. A data set belongs to an operating mode and can contain the following, for example:

| | | |
|---|---|---|
| 006F | 11 | |
| 0070 | 36 | 02 30 00 C8 00 |
| 0076 | 12 | |
| 0077 | 0F | 04 64 00 |
| 0078 | 56 | |
| 007C | 4B | 03 02 |
| 007F | 13 | |

The numbers shown in italics are line numbers that were generated while the data set 80 was being read and are not stored in the data memory 28 of the electrosurgical instrument 14; instead, they are generated by the operating program itself in accordance with the order of the references in a corresponding data set. Thus, the line numbers are the result of the order of the references (i.e. their structure) in a respective data set. The line numbers represented in the example by the numbers shown in italics are simply numbers in ascending order in accordance with the length of the structured data. The numbers shown in bold serve as references (or pointers), each of which refers to an operating specification in the generator data memory 72, namely—in the illustrated example—to control commands. Thus, “11”, for example, refers to the control command “IF”, “36” refers to a comparison that is specified by the following assigned numbers “02 30 00 C8 00”, “12” refers to the control command “THEN”, “0F” refers to a control command for setting a parameter value specified by the following assigned numbers “04 64 00”, and “56” refers to the control command “ELSE”.

The respective operating specification 76 called up by means of the parameterized reference also shows which additional information (such as “02 30 00 C8 00” in the example above) is also relevant.

The operating program 74 can read and translate the example described above as follows:
11—if->no additional information required
36—comparison->5 characters of additional information required (02=1 character type of comparison, 30 00=2 characters number 1, C8 00=2 characters number 2)
12—then->no additional information required

0F—set initial value->3 characters additional information (04=1 character which initial value; 64 00=2 characters set value)

Translated, this can mean “Compare whether number 1 (30 00) is greater than number 2 (C8 00). If the result is TRUE, set voltage to 100 V (64 00), else . . . ”

The binary numbers shown in the example as hexadecimal numbers (and herein referred to in short as “hexadecimal numbers”) in a respective data set 80 thus represent first of all references, based on which the operating program 74 can access operating specifications 76 in the generator data memory 72. The hexadecimal numbers stored in a data set in a structured (ordered) manner are the aforementioned references to operating specifications 76 stored in the generator data memory 72, such as control commands and parameters, which, due to the structure of the data set, can be assigned line numbers that, as such, do not need to be explicitly stored in the data set 80, but that can be generated by the operating program 74 during the import of a respective data set 80.

The binary numbers stored in a respective data set 80 and shown as hexadecimal numbers in the example serve as pointers, each of which refers to a specific operating specification 76 in the generator data memory 72 of the electrosurgical generator 12 so that the corresponding hexadecimal number is linked to a corresponding specific operating specification 76 in the generator data memory 72 of the electrosurgical generator 12. Thus, these hexadecimal numbers are used to designate a corresponding memory entry, representing an operating specification 76, in the generator data memory 72 of the electrosurgical generator 12. These hexadecimal numbers are therefore a kind of pointer for guiding the operating program 74 to memory entries in the generator data memory 72 of the electrosurgical generator 12, where the operating program can, during its execution, call up an operating specification for the operating program in each case. The calling up of the references is controlled by the operating program, resulting in the processor 70 calling up those memory entries in the generator data memory 72 to which the references refer. Jumps within the references that are identified by their sequence or line numbers are also possible. Via the memory entries in the generator data memory 72 corresponding to it, the structured data in a data set can thus be translated into operating specifications for the operating program.

In the data set shown by way of example above, the entries in the first column (“006F, 0070, 0076 . . . ”) are line numbers. The line numbers are not stored in the instrument data memory 28 of the electrosurgical instrument 14, but are generated by the operating program 74 itself, since the line numbers are simply numbers in ascending order in accordance with the length of the structured data:
0070 36 02 30 00 C8 00 (data length=6, i.e. the next line number is 0076)
0076 12 (data length=1, i.e. the next line number is 0077)

The entries in each line (“11, 36 02 30 00 C8 00, 12 . . . ”) refer to operating specifications 76 in the generator data memory 72. The entry in the generator data memory labeled “11” may for example be an “IF” instruction, while the entry labeled “12” may be a “THEN” instruction. The “IF” instruction and the “THEN” instruction are each one operating specification. Based on the associated memory entries in the generator data memory 72, the hexadecimal numbers “36 02 30 00 C8 00” that, in the structured data of the illustrated data set, are located between 11 and 12 can be translated into a control instruction, such as “Compare the last read value of the voltage (30 00) with the number 200 (C8 00) and return “TRUE” if the voltage is smaller than or equal to 200, else return “FALSE”. When the operating program 74 calls up the memory entries for the string “0F 04 64 00” from the generator data memory 72 at the address “0077” (generated by the operating program), this could denote a setting for the electrosurgical generator 12; the setting may e.g. be that a maximum value for the DC output voltage (0F 04) is set to 100V (64 00).

The control instructions concerning the evacuation device can, for example, define an on or off switching of the evacuation device or, for the switched-on state of the evacuation device, the respective power to be delivered by the evacuation device.

The electrosurgical generator 12 is designed to convert such control instructions, which define the power to be delivered by the evacuation device 26, into control interventions in the form of pulse sequences which, in the sense of pulse width modulation, cause the evacuation device 26 to be switched on and off in rapid succession so that it is operated with a higher or lower capacity as a result. A sequence of switch-on pulses emitted in rapid succession in the context of pulse width modulation is defined by the length of the switch-on pulses and the length of the pauses (switch-off times) between the individual switch-on pulses. The shorter the pauses (switch-off times) and the longer the switch-on pulses, the higher the power output of the evacuation unit is as a result.

The structured data 80 stored in the data structure 78 contains entries that define the control instructions that are converted into control interventions on the evacuation device 26 during operation of the electrosurgical generator 12.

In the preferred embodiment shown, the electrosurgical generator 12 is designed to control the power supplied by the evacuation device 26 during operation depending on a calculated parameter value or a parameter value detected by sensors. To this end, the electrosurgical generator 12 may have power measuring means for measuring the actual power output by the electrosurgical generator 12, and control the evacuation device 26 accordingly.

Thus, the operation of the electrosurgical generator 12, and of the evacuation device 26 connected to it, in a respective operating mode depends first of all on the operating program 74 stored in the generator data memory 72. However, in addition, the operating behavior of the electrosurgical generator 12 in a respective operating mode also depends on the data set 80 in the data structure 78 called up for a respective operating mode as well as on the operating specifications 76 stored in the generator data memory 72.

The advantage of such an electrosurgical generator 12 is that new operating modes and respective suitable activations of the evacuation device 26 can easily be defined by generating new data sets 80 in the data structure 78, and that a single operating mode can, for example, be changed solely by changing the corresponding data set 80 in the data structure 78, without the operating program 74 in the generator data memory 72 or the operating specifications 76 in the generator data memory 72 having to be changed. On the other hand, global parameters, such as any potential control instructions that are available or operating parameters depending on the respective electrosurgical generator, such as its maximum AC output voltage or a minimum permissible DC output voltage, can be stored as operating specifications 76 in the generator data memory 72, where they are, if need be, also changed centrally for all possible operating modes at the same time.

Another advantage of the design of the electrosurgical generator 12 is that a data set 80, the structured data of which indirectly defines an operating mode suitable for the electrosurgical instrument 14, can also be stored in an electrosurgical instrument 14; see FIG. 5. Specifically, the instrument data memory 28 of the electrosurgical instrument 14 can contain a data set 80 with structured data that is compatible with the data structure 78 and, just like other structured data in the data structure 78, indirectly defines a respective operating mode by means of corresponding references to operating specifications 76 in the generator data memory 72.

The electrosurgical generator 12 is thus configured in such a way that, when an electrosurgical instrument 14 is connected, the electrosurgical generator 12 will, in each case, first read the instrument data memory 28 of the electrosurgical instrument 14—if available—and enter the structured data from the data set 80 stored in the data memory 28 into the data structure 78. Thus, an operating mode precisely tailored to the respective electrosurgical instrument 14 will be available to the electrosurgical generator 12 during operation.

In order to allow access to the content of the instrument data memory 28 of the electrosurgical instrument 14, at least the data line 22 with a corresponding connection 18.3 is provided. As an alternative or in addition, a wireless interface, such as a Bluetooth interface or an NFC interface, may be provided for accessing the content of the instrument data memory 28 of the electrosurgical instrument 14.

When the structured data is transferred from the instrument data memory 28 of the electrosurgical instrument 14 into a corresponding data set in the data structure 78 of the electrosurgical generator 12, the line numbers can, if applicable, be generated to match the operating program 74 of the electrosurgical generator 12.

It is a great advantage that the data set stored in the instrument data memory 28 only contains references ordered in a structured manner (pointers to further memory entries in the generator data memory 72), but does not directly contain any control instructions or operating parameters for a respective operating mode since the control instructions and the operating parameters are centrally stored as operating specifications 76 in the generator data memory 72 of the electrosurgical generator 12.

Alternatively, the electrosurgical generator 12 can also be configured such that it directly reads out the instrument data memory 28 of the electrosurgical instrument 14 during operation—i.e. during execution of the operating program. This is the case in the example shown in FIG. 5. In this case, the structured data of the data set in the instrument data memory 28 of the electrosurgical instrument 14 does not need to be transferred into the data structure 78 of the electrosurgical generator 12 first. However, the line numbers to be generated must match the corresponding call-ups in the operating program and the entries in the generator data memory 72 in this case.

An advantage of the electrosurgical system 10 of the type described herein is that different parameter values that define the operation of the electrosurgical generator 12 and operating specifications can be managed independently of one another. Thus, the operating program 74 stored in the generator data memory 72 is stored independently of the operating specifications 76 in the generator data memory 72. Operating specifications 76 are, in turn, stored independently of the structured data in the data structure 78.

A programming interface 82 is preferably provided that preferably provides a graphical user interface, is created via a data set with structured data implemented as a plurality of parameterized references, and can be stored in the instrument data memory 26 of the electrosurgical instrument 14.

Preferably, the programming interface 82 is configured such that it assigns different rights to different users. Thus, different rights can be assigned for programming the operating program 74 that is stored in the generator data memory 72, for entering the operating specifications 76 that are also stored in the generator data memory 72, and for the structured data that is stored in the data structure 78. This way, it can in particular be ensured that changes to the operating specifications 76 or changes to the operating program 74 can only be made by developers who are familiar with the respective electrosurgical generator 12. The operating program 74 and the operating specifications 76 can thus be programmed by developers who are familiar with the respective electrosurgical generator 12, while a developer who is familiar with the electrosurgical instrument 14 can define the operating modes for an electrosurgical instrument 14 by creating a corresponding data set with structured data. Preferably, data sets created by a developer who is familiar with the electrosurgical instrument 14 are stored in the instrument data memory 28 of the respective electrosurgical instrument 14, while further operating modes can also be stored directly in the data structure 78 of the electrosurgical generator 12. To this end, the electrosurgical generator 10 can have a USB programming interface, for example. Either the data line 32 in the connection cable 16 with a corresponding interface, or—as an alternative or in addition—a wireless interface, such as a Bluetooth interface or an NFC interface, is available for the structured data stored in the instrument data memory 28 of the respective electrosurgical instrument 14. The structured data from the data set in the instrument data memory 28 of the electrosurgical instrument 14 can then be transferred into the data structure 78 of the electrosurgical generator 12 when the electrosurgical instrument 14 is connected.

This is, in particular, relevant with regard to different electrosurgical instruments 14, since the electrosurgical instruments 14 might be integrated by other developers than the electrosurgical generator 12. The developers of the electrosurgical generator 12 can store all the specific parameter data and control commands that are important for the electrosurgical generator 12—if need be in dependence on the operating program stored in the generator data memory 72—as operating specifications 76 in the generator data memory 72. Such parameter values can, for example, be maximum or minimum permissible values for the DC output voltage, the AC output voltage, etc.

Independently of this, developers of an electrosurgical instrument 14 can use the structured data in the data set in the instrument data memory 28 of the electrosurgical instrument to specify in detail how a specific operating mode can be executed for this electrosurgical instrument 14 within the framework of the threshold values defined by the operating program 74 in the generator data memory 72 and the operating specifications 76 in the generator data memory 72. The developers of the electrosurgical instrument 14 do not need to give any further consideration to the specifications provided by the operating program 74 and the operating specifications 76. Instead, the developers of the electrosurgical instrument 14 can accept these specifications provided for the respective electrosurgical generator 12.

A programming interface 82, via which a developer can fully define an operating mode for a respective electrosurgical instrument 14, is available to the developers of an electrosurgical instrument 14 for defining an operating mode for the respective electrosurgical instrument 14 and the associated control instructions for operation of the evacuation device. This definition includes, for example, all current and voltage parameter values as well as timing specifications and transition conditions for the operation of the electrosurgical instrument. This way the operating mode can be developed with the aid of an easy-to-use tool and virtually without any software development knowledge by a developer for an electrosurgical instrument. The programming interface 82 provides to the developer a number of parameter sets that the developer can use to define different phases, for example the initial incision phase, the cutting phase, the coagulation phase, but also short-circuit or power monitoring for the respective electrosurgical instrument and the matching volume flow of the evacuation device 26. A development tool belonging to the programming interface 82 generates a memory-saving set of structured data from the specifications, wherein said set of data forms a data set that can be stored in the data memory 28 of the electrosurgical instrument 14 in a non-volatile manner.

If a new operating mode for an electrosurgical instrument defined by structured data in a data set or a modified control of the evacuation device also requires a modification of the operating program in the generator data memory or of the operating specifications in the generator data memory 72, such modifications can, for example, be implemented via a programming interface by a developer who is familiar with the electrosurgical generator.

This way it can be ensured that the structured data that defines an operating mode is compatible with the operating specifications 76 in the generator data memory 72 and the operating program 74 in the generator data memory 72.

When an electrosurgical instrument 14 is connected to the electrosurgical generator 12, the electrosurgical generator 12 reads the instrument data memory 28 in the electrosurgical instrument 14 and calls up the operating specifications 76 in the generator data memory 72 that are designated by the references contained in the structured data, so that the current and voltage curves, including any timing requirements and other conditions, defined in those operating specifications 76 are applied—also with regard to the evacuation device. This allows for reduced development times and costs. For the most part, the electrosurgical instrument 14 can be integrated independently of an electrosurgical generator 12. In addition, this allows for a shorter time-to-market, since the operating modes can also be developed and finalized after the introduction of an electrosurgical generator 12. Optimizations of an operating mode and new operating modes can be easily introduced by means of updated or new electrosurgical instruments 14. An operating mode for an electrosurgical instrument can be defined with almost no software development knowledge.

If the electrosurgical system 10 is to be operated with an electrosurgical instrument 14 connected to the electrosurgical generator 12, the appropriate operating mode will already be available once the electrosurgical instrument 14 has been connected since the associated data sets 80 with the structured data can be read by the instrument data memory 28 of the electrosurgical instrument 14. Therefore, a user will, for example, only have to actuate a switch 84, in order to operate the electrosurgical instrument 14 in the appropriate operating mode of the electrosurgical generator 12. After connecting the electrosurgical instrument 14, the user does not need to set or program anything.

The switch 84 is connected to the processor 70 of the electrosurgical generator 12 via a line 86, so that the execution of the operating program 74 stored in the generator data memory 72 can be started and stopped through the actuation of the switch 84. The switch 84 may be a foot-operated switch, but may also be a hand-operated switch, that is, for example, located at the electrosurgical hand-held instrument 14. A wireless control connection can be provided instead of the line 86.

Another alternative to a switch 84 is an automatic start of the operating program, which a user can activate in advance. In this case, the electrosurgical instrument first outputs a small measurement voltage in order to detect tissue contact (current flow) with the aid of said measurement voltage. If tissue contact—i.e. current flow—is detected, the actual operating program for the electrosurgical instrument will be called up. If the tissue contact disappears, the actual operating program for the electrosurgical instrument will be ended, and a small measurement voltage will once again be output so that a new tissue contact can be detected with the aid of said measurement voltage.

Under the control of the operating program, the processor indirectly accesses individual operating specifications 76 in the generator data memory 72 during the use of an operating mode by first accessing the references in the data structure 78 and subsequently calling up the operating specification or operating specifications referred to by the respective reference.

Depending on the operating program 74 as well as on the operating specifications 76 in the generator data memory 72 and on the data structure 78 as well as on signals 90 coming from the detection units 54, 56 and 58, the processor 70 generates control signals 88 for the generator control unit 46.

### REFERENCE NUMBERS

10 electrosurgical system
12 electrosurgical generator
14 electrosurgical instrument
16 connection cable
18.1, 18.2 electrical outputs
18.3 connection
20 shaft
20.1, 20.2 outputs
22 active electrode
24 handle
26 evacuation device
27 control line
28 data memory
30.1, 30.2 supply lines
32 data line
40 high-voltage power supply
42 output
44 high-frequency part
46 output transformer
48 generator control unit
50 capacitor
52 synchronizing circuit
54 output current detection unit
56 AC output voltage detection unit
58 DC output voltage detection unit
60 high-voltage rectifier circuit
62 output capacitor
64 switch
70 processor
72 generator data memory
74 operating program
76 operating specifications
78 data structure
80 data set
84 switch
86 line
88 control signals of the processor
90 signals of the detection units

1. Electrosurgical system with an electrosurgical instrument, an evacuation device and an electrosurgical generator, to which the electrosurgical instrument and the evacuation device are connected, wherein the electrosurgical generator has a processor and at least one generator data memory and is configured to convert entries in the generator data memory into control instructions for operating the evacuation device and, during operation, to carry out control interventions corresponding to the control instructions for controlling the evacuation device, wherein the entries in the memory defining the control instructions for operating the evacuation device are specific to a respective electrosurgical instrument and/or a respective operating mode.

2. Electrosurgical system according to claim 1, wherein the generator data memory contains an operating program, operating specifications, and a data structure, of which
the operating program causes the processor to control the operation of the electrosurgical generator in conjunction with the electrosurgical instrument;
the operating specifications can be called up by the processor, which is controlled by the operating program, when the electrosurgical generator is in operation, and they can influence the operation of the electrosurgical generator, but do not define any fixed operating sequences; and
data sets with structured data are stored in the data structure that contain references to operating specifications stored in the generator data memory, which allow for calling up individual references in a targeted manner by the operating program during operation,
wherein the structured data contains entries that define control instructions for operating the evacuation device.

3. Electrosurgical system according to claim 1, wherein the electrosurgical instrument comprises a non-volatile instrument data memory that contains a data set with structured data, which data is compatible with the data structure of the electrosurgical generator and contains entries that define control instructions for controlling the evacuation device.

4. Electrosurgical system according to claim 2, wherein the processor is configured, by means of the operating program, to read specific control instructions for different electrosurgical instruments and/or different operating modes and to cause them to be converted into control interventions on the electrosurgical generator, the electrosurgical instrument, and/or the evacuation device.

5. Electrosurgical system according to claim 1, wherein the control instructions defined by entries stored in a memory for control interventions on the evacuation device are conditional control instructions that define control interventions which are dynamically dependent on respective current operating states of the electrosurgical generator and/or on currently occurring operating parameter values.

6. Electrosurgical system according to claim 1, wherein the control instructions concerning the evacuation device define an on or off switching of the evacuation device or, for the switched-on state of the evacuation device, the respective power to be delivered by the evacuation device.

7. Electrosurgical system according to claim 6, wherein the electrosurgical generator is designed to convert such control instructions, which define the power to be delivered by the evacuation device, into control interventions in the form of pulse sequences which, in the sense of pulse width modulation, cause the evacuation device to be switched on and off in rapid succession so that it is operated with a higher or lower capacity as a result.

8. Electrosurgical system according to claim 1, wherein the electrosurgical generator is designed to control the power delivered by the evacuation device during operation depending on a calculated parameter value or a parameter value detected by sensors.

9. Electrosurgical system according to claim 1, wherein the electrosurgical generator features power measuring means for measuring the power output by an electrosurgical instrument.

10. An electrosurgical system according to claim 1, with one programming interface or several programming interfaces, by means of which the operating specifications and the data structure can be programmed.

11. Method of operating an electrosurgical system according to claim 1, wherein entries in the generator data memory are converted into control instructions for operating the evacuation device and, during operation, control interventions corresponding to the control instructions for controlling the evacuation device are implemented, wherein entries defining the control instructions for operating the evacuation device in the generator data memory are specific to a respective electrosurgical instrument and/or a respective operating mode.

12. Method according to claim 11, wherein the entries defining the control instructions for operating the evacuation device are read from a data structure in an instrument data memory of an electrosurgical instrument.

13. Electrosurgical generator for an electrosurgical system according to claim 1, with a processor and at least one generator data memory, wherein the processor is configured to convert entries in the generator data memory into control instructions for operating the evacuation device and, during operation, to carry out control interventions corresponding to the control instructions for controlling the evacuation device, wherein the entries in the memory defining the control instructions for operating the evacuation device are specific to a respective electrosurgical instrument and/or a respective operating mode.

14. Electrosurgical generator according to claim 13, wherein the electrosurgical generator is designed to convert such control instructions, which define the power to be delivered by the evacuation device, into control interventions in the form of pulse sequences which, in the sense of pulse width modulation, cause the evacuation device to be switched on and off in rapid succession so that it is operated with a higher or lower capacity as a result.

15. Electrosurgical instrument for an electrosurgical system according to claim 1, with an instrument data memory containing data sets with structured data, which data contains entries defining the control instructions for operating an evacuation device.
